(19) United States
(12) Patent Application Publication
(10) Pub. No.: US 2022/0081492 A1
(43) Pub. Date: Mar. 17, 2022

(54) Chitin Whisker-Enhanced Hyaluronic Acid Cell Scaffold and Preparation Method Thereof

(71) Applicant: Hangzhou Singclean Medical Products Co., Ltd, Hangzhou (CN)

(72) Inventors: Feifei Wu, Hangzhou (CN); Jie Li, Hangzhou (CN); Jin Zeng, Hangzhou (CN); Caili Ma, Hangzhou (CN); Kui Zhou, Hangzhou (CN); Shitu Ma, Hangzhou (CN)

(21) Appl. No.: 17/106,194

(22) Filed: Nov. 30, 2020

(30) Foreign Application Priority Data
Sep. 17, 2020 (CN) 202010979595.5

(51) Int. Cl. C08B 37/08; C12M 1/12

(52) U.S. Cl. CPC C08B 37/0072; C12M 25/14; C08B 37/003

(57) ABSTRACT

Provided are a chitin whisker-enhanced hyaluronic acid cell scaffold and a preparation method thereof. Components of the cell scaffold include chitin whiskers and cross-linked hyaluronic acid. The chitin whisker-enhanced hyaluronic acid cell scaffold is obtained by dispersing chitin whiskers into deionized water using ultrasound, followed by addition of hyaluronic acid and uniform mixing to obtain an aqueous solution, adjusting a pH value of the aqueous solution to be in a range of 4.0 to 6.0, subjecting the aqueous solution to a cross-linking reaction, dialyzing a reaction product in a phosphate buffer solution, and freeze drying a resulting product. The chitin whisker-enhanced hyaluronic acid cell scaffold and the preparation method thereof can increase a mechanical property and a resistance to degradation of a scaffold material and expand an application scope of hyaluronic acid cell scaffolds.

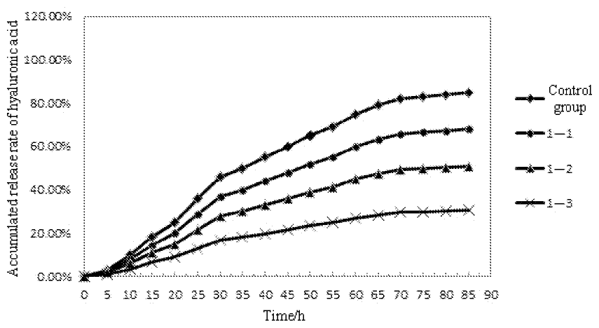

### CROSS REFERENCE TO RELATED APPLICATIONS

The present application claims the benefit of Chinese Patent Application No. 202010979595.5 filed on Sep. 17, 2020, the contents of which are incorporated herein by reference in their entirety.

### FIELD OF THE INVENTION

The present disclosure relates to the technical field of biological materials, and in particular, to an enhanced cross-linked hyaluronic acid cell scaffold and a preparation method of the enhanced cross-linked hyaluronic acid cell scaffold.

### BACKGROUND OF THE INVENTION

Hyaluronic acid (HA) is a natural mucopolysaccharide formed by alternate link of two kinds of structural units, i.e., glucuronic acid and N-acetylglucosamine, is widely present in extracellular matrix, connective tissue, and biological organs of higher animals, and thus has good biocompatibility and degradability and is an ideal tissue engineering scaffold material. However, a scaffold material prepared from cross-linked hyaluronic acid is still unsatisfactory in mechanical strength, porosity and so on.

### SUMMARY OF THE INVENTION

In order to overcome deficiencies of existing technologies, the present disclosure aims to provide a chitin whisker-enhanced hyaluronic acid cell scaffold and a preparation method of the chitin whisker-enhanced hyaluronic acid cell scaffold, so as to increase a mechanical property and a resistance to degradation of a scaffold material and expand an application scope of hyaluronic acid cell scaffolds.

According to a first aspect of the present disclosure, the present disclosure provides a chitin whisker-enhanced hyaluronic acid cell scaffold. Components of the cell scaffold include chitin whiskers and cross-linked hyaluronic acid.

The cross-linked hyaluronic acid is reduction-responsive cross-linked hyaluronic acid, preferably cross-linked hyaluronic acid containing disulfide bonds. A mass ratio of the chitin whiskers to the hyaluronic acid which is a raw material added before cross-linking is 0.01:100 to 30:100.

According to a second aspect of the present disclosure, the present disclosure provides a preparation method of a chitin whisker-enhanced hyaluronic acid cell scaffold. The method includes the following steps of:

dispersing chitin whiskers into deionized water using ultrasound, followed by addition of hyaluronic acid and uniform mixing to obtain an aqueous solution; adjusting a pH value of the aqueous solution to be in a range of 4.0 to 6.0, and then subjecting the aqueous solution to a cross-linking reaction; and performing dialyzing a resulting reaction product in a phosphate buffer solution, and freeze drying a resulting product to obtain the chitin whisker-enhanced hyaluronic acid cell scaffold.

Preferably, the method includes the following steps of: dispersing chitin whiskers into deionized water using ultrasound, followed by addition of hyaluronic acid and uniform mixing to obtain an aqueous solution; adjusting a pH value of the aqueous solution to be in a range of 4.0 to 6.0, followed by successive addition of 1-(3-dimethylaminopropyl)-3-ethylcarbodiimide hydrochloride (EDC.HCl) and N-hydroxysuccinimide (NHS), uniform mixing, and then addition of an aqueous solution of cystamine dihydrochloride; readjusting a pH value of a resulting reaction system to be in a range of 4.0 to 6.0, and subjecting the reaction system to a reaction; dialyzing a resulting reaction product in a phosphate buffer solution, and freeze drying a resulting product to obtain the chitin whisker-enhanced hyaluronic acid cell scaffold.

In the preparation method of a chitin whisker-enhanced hyaluronic acid cell scaffold, chitin whiskers and non-cross-linked hyaluronic acid are mixed, and then after a cross-linking reaction and processes such as freeze drying, a cell scaffold material is prepared. This method enables the chitin whiskers to be dispersed in a hyaluronic acid hydrogel more uniformly.

Further, the cell scaffold material has a good mechanical strength, including a tensile strength, a compressive strength, and an elastic modulus.

Further, a mass ratio of the chitin whiskers to the hyaluronic acid is in a range of 0.01:100 to 30:100.

Further, a temperature of the reaction is maintained in a range of 0° C. to 50° C., preferably in a range of 5° C. to 10° C.

Further, a time length of the reaction is in a range of 12 h to 144 h, preferably 48 h to 60 h.

Further, a time length of the dialyzing is in a range of 6 h to 72 h, preferably 24 h to 36 h.

Chitin whiskers (CW) are chitin fibers existing in a single crystal form. The inventor thinks that the chitin whiskers have a relatively high modulus due to the regular arrangement of crystal grains and few internal defects, a transverse modulus and a longitudinal modulus thereof being respectively as high as 15 GPa and 150 GPa. The chitin whiskers retain the structure and biological activity of chitin, have biodegradability, a unique antibacterial property and good biocompatibility, and thus are suitable for use as a reinforcing material. The present disclosure enhances the cross-linked hyaluronic acid containing disulfide bonds by adding chitin whiskers, and the chitin whisker-enhanced hyaluronic acid cell scaffold material has a better mechanical strength and a better resistance to degradation; and moreover, cell scaffolds that are different in mechanical strength, pore size, and resistance to degradation can be obtained by changing a ratio of a chitin whisker suspension to the cross-linked hyaluronic acid. The cell scaffolds can be used in tissue engineering scaffolds for cartilage regeneration, leukoderma treatment, artificial skin and so on.

### BRIEF DESCRIPTION OF THE DRAWINGS

The sole FIGURE is a line graph showing changes in releasing of hyaluronic acid by cell scaffolds over time in processes of Examples 1 to 3 of the present disclosure and a Control Group (a cross-linked hyaluronic acid scaffold material containing no chitin whiskers).

### DETAILED DESCRIPTION OF THE EMBODIMENTS

In order to make the present disclosure better understood, the chitin whisker-enhanced hyaluronic acid cell scaffold provided by the present disclosure is described in detail below in conjunction with embodiments. However, the present disclosure is not limited to these embodiments, and non-essential improvements and adjustments made by those skilled in the art under the core concept of the present disclosure still fall into the protection scope of the present disclosure.

(I). Preparation of a Chitin Whisker-Enhanced Cell Scaffold Material

### Example 1

(1) Preparation of chitin whiskers: Shrimp and crab shells were crushed into powders and dispersed into concentrated sulfuric acid of 3M, with a mixing ratio of chitin to a concentrated sulfuric acid solution being 3% w/v, followed by stirring for 12 h in a water bath at 90° C. A resulting mixture was diluted with deionized water and then subjected to centrifugation to remove a supernatant liquor, which was repeated three times. A resulting precipitate was re-suspended with deionized water. A resulting suspension liquid was transferred to a dialysis bag with a molecular weight cut-off in a range of 8000 Da to14000 Da and sufficiently dialyzed until a pH value of the suspension liquid in the dialysis bag became 6.0. The suspension liquid was then subjected to ultrasonic treatment at a power of 600 W for 10 min so that the precipitate suspended completely. Centrifugation was performed to a resulting suspension liquid at a speed of 7200 rpm, and then freeze drying was performed to obtain chitin whiskers (reference can be made to CN 110478523 A).

(2) Preparation of a chitin whisker-enhanced hyaluronic acid cell scaffold: 0.01 g of chitin whiskers were dispersed into 100 mL of deionized water using ultrasound, followed by addition of 1 g of hyaluronic acid to obtain an aqueous solution. The aqueous solution was adjusted to have a pH value of 5.0, followed by successive addition 0.5 g of 1-(3-dimethylaminopropyl)-3-ethylcarbodiimide hydrochloride (EDC.HCl) and 0.3 g of N-hydroxysuccinimide (NHS), uniform stirring at room temperature, and then addition of an aqueous solution of cystamine dihydrochloride after 6 h. A resulting reaction system was readjusted to have a pH value in a range of 5.0 to 6.0, and then subjected to a reaction under stirring at 10° C. for 60 h. After that, dialysis was performed to a reaction product in a phosphate buffer solution with pH=7.0 for 36 h, and freeze drying was performed at −80° C. for 48 h to obtain a cross-linked hyaluronic acid cell scaffold (First Group: 1-1).

### Example 2

Except that a mass of the chitin whiskers was changed to be 2% of that of the hyaluronic acid, other steps were the same as those in Example 1 (Second Group: 1-2).

### Example 3

Except that a mass of the chitin whiskers was changed to be 5% of that of the hyaluronic acid, other steps were the same as those in Example 1 (Third Group: 1-3).

### Example 4

Except that no chitin whiskers were added, other steps were the same as those in Example 1 (Control Group).

(II). Measurement of a Thickness and a Pore Size of a Chitin Whisker-Enhanced Cross-Linked Hyaluronic Acid Cell Scaffold

Method: A small piece was taken by cutting from the scaffold material obtained in each of Examples 1 to 4. A thickness of the material was measured with a vernier caliper, and a pore size thereof was measured by a scanning electron microscope.

Results indicated that, thicknesses and pore sizes of scaffold materials, in which chitin whiskers were added, were improved to a certain extent, and a suitable pore size was beneficial to exchange of nutrient substances required for growth of cells and to migration and proliferation of cells.

TABLE 1
Thicknesses and pore sizes of chitin whisker-enhanced
cell scaffolds obtained in Examples 1 to 4

| | Mass Ratio | Material Thickness | Pore Size |
|---|---|---|---|
| Sample Number | (CW:HA) | (mm) | (μm) |
| Control Group | 0:100 | 1.14 ± 0.21 | 103 ± 16 |
| Group 1-1 | 1:100 | 1.22 ± 0.36 | 131 ± 18 |
| Group 1-2 | 2:100 | 1.35 ± 0.47 | 148 ± 25 |
| Group 1-3 | 5:100 | 1.67 ± 0.95 | 172 ± 51 |

(III) Measurement of a Mechanical Strength of a Chitin Whisker-Enhanced Hyaluronic Acid Cell Scaffold

Method: A small piece of 3 cm×3 cm was taken by cutting from the cross-linked hyaluronic acid scaffold material obtained in each of Examples 1 to 4. A tensile property of the obtained material was measured with a tensile machine.

Results indicated that, tensile strengths of scaffold materials, in which chitin whiskers were added, were improved to various degrees; and moreover, the higher a content of the chitin whiskers was, the higher the mechanical strength of the obtained scaffold material was.

TABLE 2
Tensile strengths of chitin whisker-enhanced
cell scaffolds obtained in Examples 1 to 4

| Sample Number | Mass Ratio (CW:HA) | Tensile strength (N) |
|---|---|---|
| Control Group | 0:100 | 8.2 ± 0.8 |
| Group 1-1 | 1:100 | 14.2 ± 1.3 |
| Group 1-2 | 2:100 | 16.2 ± 1.4 |
| Group 1-3 | 5:100 | 20.2 ± 1.9 |

(IV) Measurement of an Expansion Rate and a Water Absorption Rate of a Chitin Whisker-Enhanced Cross-Linked Hyaluronic Acid Cell Scaffold

Method for measuring an expansion rate: A small piece of 3 cm×3 cm was taken by cutting from the cross-linked hyaluronic acid cell scaffold prepared in each of Examples 1 to 4. The small piece was added into 30 mL of a sodium chloride solution with a concentration of 0.9%, and the sodium chloride with the small piece was then placed into a water bath at 37° C. for 2.5 h. After that, a length and a width of the small piece were measured. An expansion rate was a percentage of a length multiplied by a width after swelling to a length multiplied by a width before the swelling.

Method for measuring a water absorption rate: A small piece of 2 cm×2 cm was taken by cutting from the cross-linked hyaluronic acid cell scaffold prepared in each of Examples 1 to 4, and was weighed to obtain a value which was marked as W₁. The small piece was added into 20 mL of a sodium chloride solution with a concentration of 0.9% at 37° C. After being placed for 10 min, the small piece was taken out with tweezers. Unnecessary moisture on the surface of the small piece was wiped up, and then the small piece was weighed to obtain a value which was marked as W₂. A water absorption rate is specific value of a weight of moisture absorbed by the scaffold material within a certain period of time to a weight of the scaffold material itself.

Results indicated that the expansion rate and the water absorption rate were not reduced due to the addition of chitin whiskers, and moreover the expansion rate and the water absorption rate were increased slightly.

TABLE 3
Expansion rates and water absorption rates of chitin whisker-
enhanced cell scaffolds obtained in Examples 1 to 4

| | Mass Ratio | Expansion Rate | Water Absorption |
|---|---|---|---|
| Sample Number | (CW:HA) | (%) | Rate (%) |
| Control Group | 0:100 | 98.5 ± 4.6 | 56.9 ± 3.1 |
| Group 1-1 | 1:100 | 100.5 ± 3.5 | 58.3 ± 1.8 |
| Group 1-2 | 2:100 | 101.5 ± 2.7 | 59.5 ± 2.5 |
| Group 1-3 | 5:100 | 102.8 ± 6.5 | 61.2 ± 2.7 |

(V) Measurement of Cytotoxicity of a Chitin Whisker-Enhanced Cross-Linked Hyaluronic Acid Cell Scaffold

A cytotoxicity test was performed to the cross-linked hyaluronic acid cell scaffold prepared in each of Examples 1 to 4.

The measurement was performed in accordance with Part 5 Tests for in vitro cytotoxicity in Biological evaluation of medical devices. The obtained chitin whisker-enhanced cell scaffold material was cut into pieces. 1 g of pieces was added into 1 mL of a cell culture fluid, and the cell culture fluid with the pieces was placed at 37±2° C. for 30 h. A leach liquor was diluted with a culture medium, so as to obtain a series of diluted leach liquors as sample solutions. Subsequently, cytotoxicity of the scaffold material was evaluated with MTT assay.

Results indicated that the chitin whisker-enhanced hyaluronic acid cell scaffold had no cytotoxicity and had good biocompatibility.

TABLE 4
Cytotoxicity of chitin whisker-enhanced cell
scaffolds obtained in Examples 1 to 4

| Sample Number | Mass Ratio (CW:HA) | Cytotoxicity Reaction |
|---|---|---|
| Control Group | 0:100 | Grade 0 |
| Group 1-1 | 1:100 | Grade 0 |
| Group 1-2 | 2:100 | Grade 0 |
| Group 1-3 | 5:100 | Grade 0 |

(VI) Measurement of a Degradation Property of a Chitin Whisker-Enhanced Cross-Linked Hyaluronic Acid Cell Scaffold

Method: The scaffold materials obtained in Examples 1 to 4 were cut into pieces. Three parts, each having 10 mg of pieces, were measured for each of the scaffold materials, and were added into a PBS buffer solution (pH=7.4) having 2 mM of GSH. The PBS buffer solution with the pieces was placed into a water bath at a constant temperature of 37° C. and was stirred magnetically for reaction. After a period of time, dissociative hyaluronic acid dissociated from the cell scaffold was detected by taking 1 mL of a supernatant liquor and diluting the supernatant liquor for measurement of a content of uronic acid.

Results were shown in the sole FIGURE, and indicated that the cell scaffold could be gradually dissociated over time under a reduction condition until the cell scaffold was changed into a solution state and that a resistance to degradation of the scaffold materials was improved by addition of the chitin whiskers. A degradation time length of the material could be adjusted to some degree as required by adjusting a dosage of the chitin whiskers.

The above results indicated that the chitin whisker-enhanced hyaluronic acid cell scaffold material prepared by the method of the present disclosure has good biocompatibility and is excellent in the pore size, the expansion rate and the water absorption rate; and moreover the mechanical property and the resistance to degradation of the scaffold are improved distinctly due to addition of the chitin whiskers. Therefore, the cell scaffold of the present disclosure can be used in cell engineering and tissue regeneration engineering.

The above embodiments are only for the purpose of illustrating principles of the present disclosure, rather than limiting the present disclosure. Various changes and modifications can be made within the spirit and scope defined by the claims of the present disclosure, but such changes and modifications all fall into the protection scope of the present disclosure.

1. A preparation method of a chitin whisker-enhanced hyaluronic acid cell scaffold, wherein the method comprises the following steps of:
dispersing chitin whiskers into deionized water using ultrasound, followed by addition of hyaluronic acid and uniform mixing to obtain an aqueous solution; adjusting a pH value of the aqueous solution to be in a range of 4.0 to 6.0, and then subjecting the aqueous solution to a cross-linking reaction; and dialyzing a resulting reaction product in a phosphate buffer solution, and freeze drying a resulting product to obtain the chitin whisker-enhanced hyaluronic acid cell scaffold.

2. The preparation method according to claim 1, wherein the cross-linked hyaluronic acid is reduction-responsive cross-linked hyaluronic acid.

3. The preparation method according to claim 2, wherein the cross-linked hyaluronic acid is a cross-linked hyaluronic acid containing disulfide bonds.

4. The preparation method according to claim 1, wherein a mass ratio of the chitin whiskers to the hyaluronic acid is in a range of 0.01:100 to 30:100.

5. The preparation method according to claim 1, wherein a temperature of the reaction is maintained in a range of 0° C. to 50° C.

6. The preparation method according to claim 5, wherein the temperature of the reaction is maintained in a range of 5° C. to 10° C.

7. The preparation method according to claim 1, wherein a time length of the reaction is in a range of 12 h to 144 h.

8. The preparation method according to claim 7, wherein the time length of the reaction is in a range of 48 h to 60 h.

9. The preparation method according to claim 1, wherein a time length of the dialyzing is in a range of 6 h to 72 h.

10. The preparation method according to claim 9, wherein the time length of the dialyzing is in a range of 24 h to 36 h.
